(19) United States
(12) Patent Application Publication
(10) Pub. No.: US 2011/0054292 A1
(43) Pub. Date: Mar. 3, 2011

(54) SYSTEM FOR PHOTOACOUSTIC IMAGING AND RELATED METHODS

(75) Inventors: Desmond Hirson, Thornhill (CA); James I. Mehi, Thornhill (CA); Andrew Needles, Toronto (CA)

(73) Assignee: VisualSonics Inc., Toronto (CA)

(21) Appl. No.: 12/771,623

(22) Filed: Apr. 30, 2010

Related U.S. Application Data
(60) Provisional application No. 61/174,571, filed on May 1, 2009.

(51) Int. Cl. A61B 5/05

(52) U.S. Cl. 600/407

(57) ABSTRACT

Photoacoustic imaging systems and methods that allow for the creation of three-dimensional (3D) images of a subject are described herein. The systems include one or more optical fibers attached to an ultrasound transducer. Ultrasonic waves are generated by laser light emitted from the optical fiber(s) and detected by the ultrasound transducer. 3D images are acquired by ultrasound signals from a series of adjacent scan planes or frames that are then stacked together to create 3D volume data.

### CROSS REFERENCE TO RELATED APPLICATIONS

This application claims the benefit of the filing date of U.S. provisional patent application 61/174,571, filed May 1, 2009.

### FIELD OF THE INVENTION

The present invention generally relates to the fields of photoacoustic imaging and medical diagnostics. More specifically, the present invention relates to a photoacoustic imaging system that includes an ultrasound transducer with an integrated optical fiber laser that can be used to obtain three-dimensional (3D) photoacoustic images of a subject, such as a human or small laboratory animal, for diagnostic and other medical or research purposes.

### BACKGROUND

Ultrasound-based imaging is a common diagnostic tool used by medical professionals in various clinical settings to visualize a patient's muscles, tendons and internal organs, as well as any pathological lesions that may be present, with real time tomographic images. Ultrasonic imaging is also used by scientists and medical researchers conducting in vivo studies to assess disease progression and regression in test subjects.

Ultrasound imaging systems typically have a transducer that sends and receives high frequency sounds waves into the subject. The transducer often utilizes a piezoelectric component that is able to convert received ultrasound waves into an electrical signal. A central processing unit powers and controls the systems components, processes signals received from the transducer to generate images, and displays the images on a monitor.

Ultrasound imaging is relatively quick and inexpensive, and is less invasive with fewer potential side effects than other types of imaging such as X-Ray and MRI. However, conventional ultrasound technology has limitations that make it unsuitable for some applications. For example, ultrasound waves do not pass well through certain types of tissues and anatomical features, and ultrasound images typically have weaker contrast and lower spatial resolution than X-Ray and MRI images. Also, ultrasonic imaging has difficulties distinguishing between acoustically homogenous tissues (i.e. tissues having similar ultrasonic properties).

Photoacoustic imaging is a modified form of ultrasound imaging that is based on the photoacoustic effect, in which the absorption of electromagnetic energy, such as light or radio-frequency waves, generates acoustic waves. In photoacoustic imaging, laser pulses are delivered into biological tissues (when radio frequency pulses are used, the technology is usually referred to as thermoacoustic imaging). A portion of the delivered energy is absorbed by the tissues of the subject and converted into heat. This results in transient thermoelastic expansion and thus wideband (e.g. MHz) ultrasonic emission. The generated ultrasonic waves are then detected by ultrasonic transducers to form images. Photoacoustic imaging has the potential to overcome some of the problems of pure ultrasound imaging by providing, for example, enhanced contrast and spatial resolution. At the same time, since non-ionizing radiation is used to generate the ultrasonic signals, it has fewer potentially harmful side effects than X-Ray imaging or MRI.

One of the limitations of current photoacoustic systems is that none of them offers a completely satisfactory means for obtaining three-dimensional (3D) images. Attempts have been made to generate three-dimensional (3D) photoacoustic images using a tomographic approach to capture volume data using multiple ultrasound transducers arrange in a specific way or moving the single transducer around the target. These techniques typically require the subject to be immersed in water. Although systems have been developed that use a linear ultrasound transducer and laser to generate images without requiring the subject to be immerse in water, the systems typically generate only two-dimensional (2D) images.

In view of the limitations of current photoacoustic imaging methods, there remains a need for photoacoustic systems and techniques that provide an easy and convenient approach for obtaining three-dimensional (3D) photoacoustic images.

### SUMMARY OF THE INVENTION

The present invention features a photoacoustic imaging system that can be used to obtain two-dimensional (2D) or three-dimensional (3D) images of a subject. The system includes (a) an ultrasound transducer for receiving ultrasound waves, (b) a laser system for generating pulses of non-ionizing laser light, and (c) a fiber optic cable having a plurality of optical fibers attached to the transducer for directing the laser light to a target. In one embodiment, the ultrasound transducer is an arrayed transducer that has a plurality of transducer elements for generating and receiving ultrasound waves. Suitable arrayed transducers include, for example, linear array transducers, phased array transducers, two-dimensional array transducers, and curved array transducers.

The system may also include a motor for moving the ultrasound transducer. For example, the motor may be a linear stepper motor for moving the transducer along a linear path to collect a series of frames separated by a predetermined step size, which may be adjusted by the user. Typically, the step size is at least about 10 μm up to about 250 μm.

The system may also include a beamformer for receiving ultrasound signals from the transducer and focusing them along an ultrasound line. In addition, the optical fibers may be positioned on the transducer so that the laser light delivered to a subject is aligned with the ultrasound line and/or each line within a scan plane receives about the same level of laser light intensity.

In another embodiment of the invention, the photoacoustic system includes (a) a scan head having a moving support arm, (b) an ultrasound transducer, located at an end of said support arm, for receiving ultrasound waves, (c) a laser system for generating pulses of non-ionizing laser light, and (d) at least one optical fiber, more typically a plurality of optical fibers, attached to the transducer for directing the laser light to a target. The support arm is used to mechanically move the transducer along a scan plane. A separate motor may be used to move the transducer assembly in a plane perpendicular to the scan plane for obtaining a series of frames to generate 3D volume data. Alternatively, a single 2D motor may be used to move the transducer in both directions.

The various systems of the invention also typically include a central processing unit, e.g. a computer, for controlling system components and processing received ultrasound data into an image, and a monitor for displaying the image. The computer system may be equipped with software for controlling the various components according to instructions received from the user, and for visualizing and/or rendering received ultrasound data.

In another aspect, the invention features a method for generating a 3D photoacoustic image of a subject. The method includes the following steps:

(a) delivering laser radiation to a region of tissue within the subject to generate ultrasound signals for a frame;

(b) detecting the ultrasound signals for the frame;

(c) delivering laser radiation to an adjacent region of tissue to generate ultrasound signals for a next frame;

(d) detecting the ultrasound signals for the next frame;

(e) repeating steps (c) and (d) to generate a series of consecutive frames;

(f) stacking the series of consecutive frames to generate a three-dimensional volume of data; and

(g) displaying a three-dimensional image generated from the volume of data on a monitor.

When the system includes an array transducer, the ultrasound lines for the frame may be generated by a method having the following steps:

(i) positioning an aperture on the array transducer to a first line in the frame;

(ii) delivering laser radiation to the subject for the first line in the frame;

(iii) acquiring ultrasound signals for the first line in the frame;

(iv) positioning the aperture on the array transducer to a next line in the frame;

(v) delivering laser radiation to the subject for the next line in the frame;

(vi) acquiring ultrasound signals for the next line in the frame; and

(vii) repeating steps (iv) through (vi) for each subsequent line in the frame until a desired number of lines for the frame have been acquired.

A beamformer is typically used to position the aperture on the array transducer to acquire each line of the frame, and when each frame is complete a motor moves the transducer into position to acquire the lines for the next frame. The number of lines for the frame is typically from about 10 to about 1024, more typically from about 256 to about 512, and most typically is 256.

The photoacoustic imaging system and methods of the invention may be used to image various organs (e.g., heart, kidney, brain, liver, blood, etc.) and/or tissue of a subject, or to image a neo-plastic condition or other disease condition of the subject. Typically the subject is a mammal, such a human. The invention is also particularly well-suited for imaging small animals, such as laboratory mice and/or rats.

The above summary is not intended to describe each embodiment or every implementation of the invention. Other embodiments, features, and advantages of the present invention will be apparent from the following detailed description thereof, from the drawings, and from the claims. It is to be understood that both the foregoing summary and the following detailed description are exemplary and explanatory only and are not restrictive of the invention as claimed.

### BRIEF DESCRIPTION OF THE DRAWINGS

The invention may be more completely understood in consideration of the accompanying drawings, which are incorporated in and constitute a part of this specification, and together with the description, serve to illustrate several embodiments of the invention:

FIG. 1 is a top view of an ultrasound transducer with a fiber optic bundle attached to it;

FIG. 2 is a perspective view of an arrayed transducer attached to a motor stage with optical fibers attached to the transducer;

FIG. 3 is schematic diagram showing the stacking of frames into a three-dimensional (3D) volume;

FIG. 4 is a photoacoustic scan shown as a three-dimensional (3D) volume;

FIG. 5 is a block diagram showing an embodiment of a photoacoustic imaging system according to the invention, which includes an ultrasound system and a laser system with a laser cable that is integrated onto the ultrasound transducer; and

FIG. 6 is a block diagram showing the work flow of a method of photoacoustic imaging according to one embodiment of the invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The present invention provides a photoacoustic imaging system and method that allows for the creation of three-dimensional (3D) photoacoustic images of a subject. The system includes both a laser system for generating ultrasonic waves in the tissues and/or organs of the subject, and an ultrasound system that detects these ultrasonic waves and processes the received data into three-dimensional images of regions of interest within the subject.

The laser system may be, for example, a Rainbow NIR Integrated Tunable Laser System from OPOTEK California that generates non-ionizing laser pulses. The laser system also includes one or more optical fibers for delivering the laser light to the target. The optical fibers are attached to the transducer of the ultrasound system. The transmission of laser pulses into the subject results in the absorption of electromagnetic radiation, which creates ultrasonic waves. The transducer detects the ultrasonic waves generated by the laser and sends them to a central processing unit that uses software to create two-dimensional and three-dimensional images of the subject, which are displayed on a monitor.

The integration of the optical fiber laser into the ultrasound transducer allows for both ultrasound imaging and photoacoustic imaging using the same device. When obtaining the photoacoustic images the ultrasound transducer is used primarily as a detector, but the transducer can be used to both send and receive ultrasound if the user wishes to operate the device in a purely ultrasound mode. Thus the system can, in some implementations, function as both a photoacoustic imaging system as well as an ultrasound imaging system.

The ultrasound transducer can be either a single transducer system or an arrayed transducer system. In single transducer system, a swing arm or similar device is used to mechanically move the transducer along a scan plane. In arrayed transducer systems, the transducers are typically “fixed” transducers that acquire ultrasound lines in a given scan plan without the need for the transducer to be physically moved along the scan plane.

More specifically, the term “fixed” means that the transducer array does not utilize movement in its azimuthal direction during transmission or receipt of ultrasound in order to achieve its desired operating parameters, or to acquire a frame of ultrasound data. Moreover, if the transducer is located in a scan head or other imaging probe, the term “fixed” may also mean that the transducer is not moved in an azimuthal or longitudinal direction relative to the scan head, probe, or portions thereof during operation. A “fixed” transducer can be moved between the acquisitions of ultrasound frames, for example, the transducer can be moved between scan planes after acquiring a frame of ultrasound data, but such movement is not required for their operation. One skilled in the art will appreciate, however, that a “fixed” transducer can be moved relative to the object imaged while still remaining fixed as to the operating parameters. For example, the transducer can be moved relative to the subject during operation to change position of the scan plane or to obtain different views of the subject or its underlying anatomy. Indeed, as explained in more detail below, in some embodiments of the invention, a fixed transducer is attached to motor that moves its along a path perpendicular to the scan plane of the transducer to collect a series of adjacent ultrasound frames.

Examples of arrayed transducers include, but are not limited to, a linear array transducer, a phased array transducer, a two-dimensional (2-D) array transducer, or a curved array transducer. A linear array is typically flat, i.e., all of the elements lie in the same (flat) plane. A curved linear array is typically configured such that the elements lie in a curved plane.

The transducer typically contains one or more piezoelectric elements, or an array of piezoelectric elements which can be electronically steered using variable pulsing and delay mechanisms. Suitable ultrasound systems and transducers that can be used with photoacoustic system of the invention include, but are not limited to those systems described in U.S. Pat. No. 7,230,368 (Lukacs et al.), which issued on Jun. 12, 2007; U.S. Patent Application Publication No.: US 2005/0272183 (Lukacs, et al.), which published Dec. 8, 2005; U.S. Patent Application Publication No. 2004/0122319 (Mehi, et al.), which published on Jun. 24, 2004; U.S. Patent Application Publication No. 2007/0205698 (Chaggares, et al.), which published on Sep. 6, 2007; U.S. Patent Application Publication No. 2007/0205697 (Chaggares, et al.), which published on Sep. 6, 2007; U.S. Patent Application no. 2007/0239001 (Mehi, et al.), which published on Oct. 11, 2007; U.S. Patent Application Publication No. 2004/0236219 (Liu, et al.), which published on Nov. 25, 2004; each of which is fully incorporated herein by reference.

A transducer used in the system can be incorporated into a scan head to aid in the positioning of the transducer. The scan head can be hand held or mounted to rail system. The scan head cable is typically flexible to allow for easy movement and positioning of the transducer.

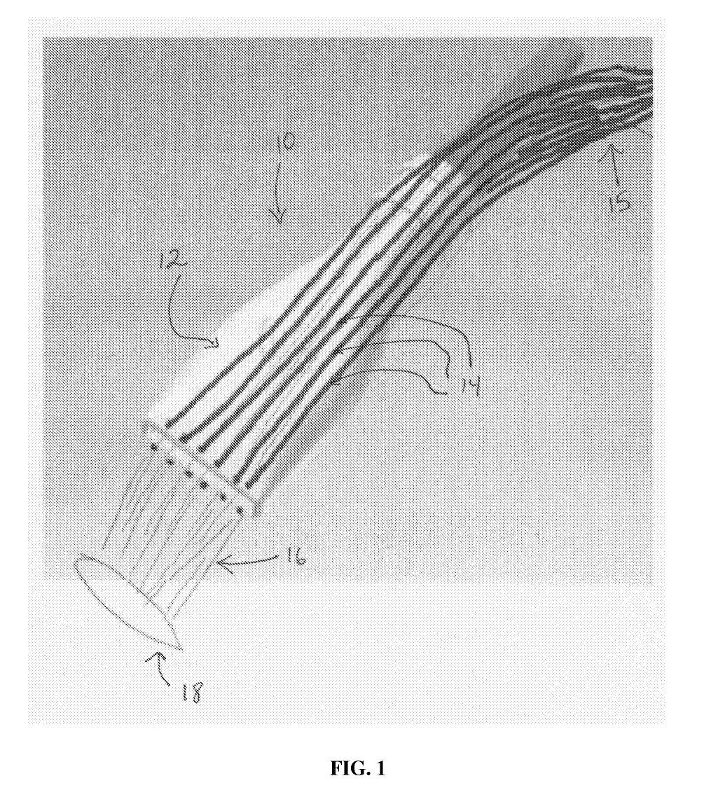

FIG. 1 shows a scan head 10 that can be used for photoacoustic imaging according to the invention. The scan head 10 has an ultrasound transducer 12 and a fiber optic cable 15 composed of a plurality of optical fibers 14, which are attached to the transducer 12. The optical fibers 14 direct laser light 16 onto the target to generate ultrasonic waves which are detected by the transducer 12. The laser light 16 emitted from the optical fibers 14 travels to an illumination region 18 on the skin surface of the subject to be imaged, and generate ultrasonic waves within the tissues of the subject.

The optical fibers and resulting light beams can be placed at different angles relative to the tissue for illumination. The angle can be increased up to 180 degrees such that the light beam delivered to subject is in-line with the ultrasound beam.

The photoacoustic images are typically formed by multiple pulse-acquisition events. Regions within a desired imaging area are scanned using a series of individual pulse-acquisition events, referred to as “A-scans” or ultrasound “lines.” Each pulse-acquisition event requires a minimum amount of time for the pulse of electromagnetic energy transmitted from the optical fibers to generate ultrasonic waves in the subject which then travel to the transducer. The image is created by covering the desired image area with a sufficient number of scan lines to provide a sufficient detail of the subject anatomy can be displayed. The number of and order in which the lines are acquired can be controlled by the ultrasound system, which also converts the raw data acquired into an image. Using a combination of hardware electronics and software instructions in a process known as “scan conversion,” or image construction, the photoacoustic image obtained is rendered so that a user viewing the display can view the subject imaged.

In one implementation of the invention, the ultrasound signals are acquired using receive beamforming methods such that the received signals are dynamically focused along an ultrasound line. The optical fibers are arranged such that each ultrasound line within the scan plane receives the same level of laser pulse intensity. A series of successive ultrasound lines are acquired to form a frame. For example, 256 ultrasound lines may be acquired, with the sequence of events for each line being the transmission of a laser pulse followed by the acquisition of ultrasound signals.

Line based image reconstruction methods are described in U.S. Pat. No. 7,052,460 issued May 30, 2006 and entitled “System for Producing an Ultrasound Image Using Line Based Image Reconstruction,” and in U.S. Patent Application Publication No. 2004/0236219 (Liu, et al.), which published on Nov. 25, 2004, each of which is incorporated fully herein by reference and made a part hereof. Such line based imaging methods image can be incorporated to produce an image when a high frame acquisition rate is desirable, for example when imaging a rapidly beating mouse heart.

For 3D image acquisition, a motor stage is typically used to move to move the ultrasound transducer with integrated fiber optic bundle in a linear motion to collect a series of frames separated by a predefined step size. The motor's motion range and step size may be set and/or adjusted by the user. Typically the step size is from about 10 μm to about 250 μm.

When mounted on a linear stepper motor, a linear array can capture a series of 2D images that are parallel to each other and spaced appropriately. Thus, the motor typically moves the array transducer along a plane that runs perpendicular to the scan plane. These 2D images are then stacked and visualized as a volume using the standard 3D visualization tools.

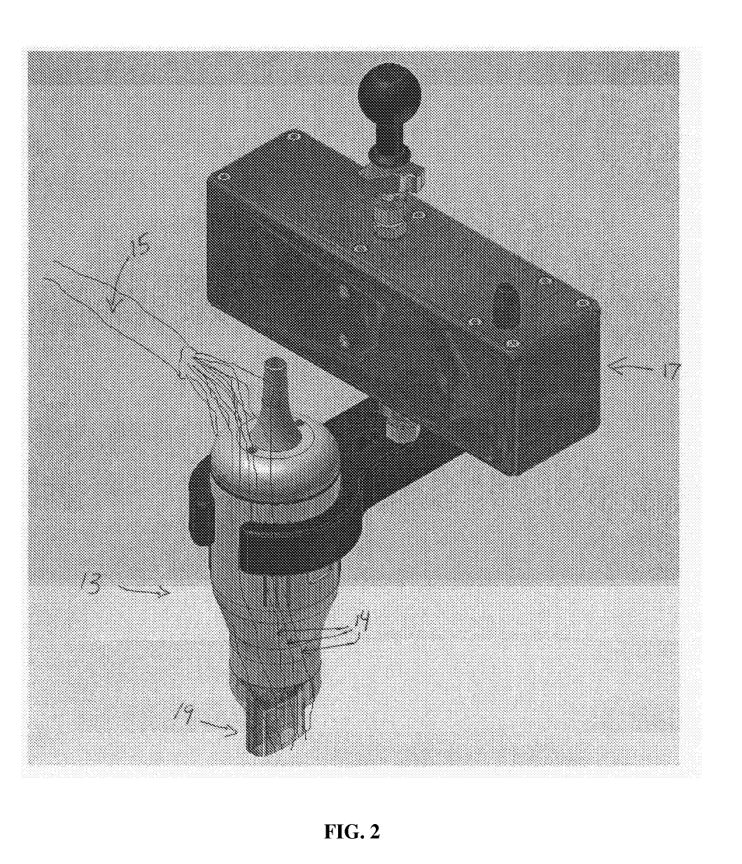

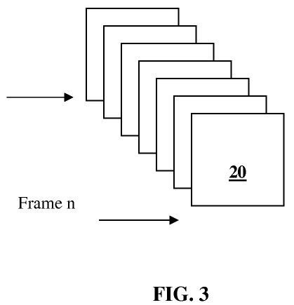

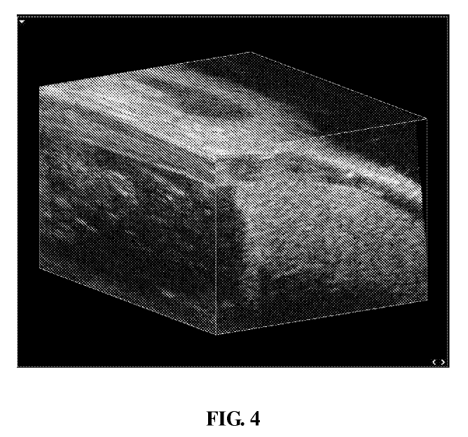

FIG. 2 shows a transducer 13 attached to a motor 17 that moves the transducer 13 along a desired path. A fiber optic cable 15 transmits laser light through a plurality of optical fibers 14 that are attached to the nosepiece 19 of the transducer 13. As the motor 17 moves the transducer 13 from one position to the next along its path, the transducer 13 acquires a series of consecutive frames (or slices) in the direction of motor travel. As shown in FIG. 3, the resulting series of frames 20 are stacked together and presented as a 3-dimensional volume of data. 3D visualization software assembles the acquired frames and renders them into a data volume or data cube. An example of a 3D data volume image is shown in FIG. 4.

For implementations of the invention using a single element transducer that is mechanically moved by a motorized swing arm or similar device along a scan plane, 3D images can also be obtained by providing the system with means for moving the transducer in the plane perpendicular to that of the scan plane. This could be either a second motor positioning system used to move the entire transducer assembly (or RMV) in the other plane for 3D acquisition, or it could be a 2D motor positioning system that moves the transducer in two different dimensions with one support arm.

In addition to an ultrasound transducer with integrated fiber optic laser and a motor for moving the transducer, as described above, photoacoustic systems according to the invention typically have one or more of the following components: a processing system operatively linked to the other components that may be comprised of one or more of signal and image processing capabilities; a digital beamformer (receive and/or transmit) subsystems; analog front end electronics; a digital beamformer controller subsystem; a high voltage subsystem; a computer module; a power supply module; a user interface; software to run the beamformer and/or laser; software to process received data into three-dimensional (3D) images; a scan converter; a monitor or display device; and other system features as described herein.

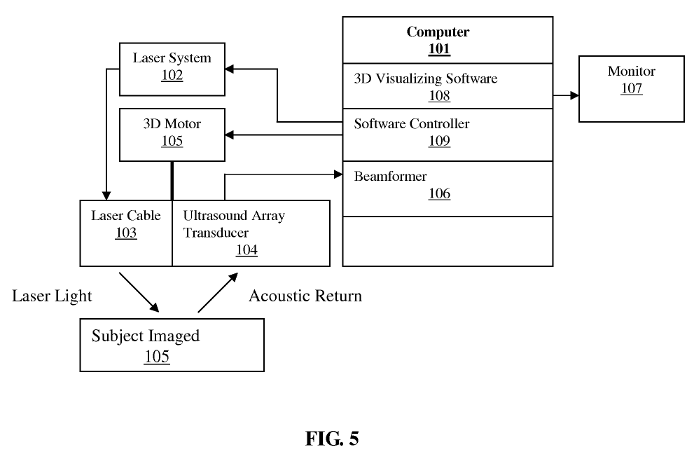

FIG. 5 is a block diagram illustrating an exemplary photoacoustic imaging system of the invention. The system includes an array transducer 104 with integrated fiber optic cable 103 for directing laser light generated by the laser system 102 onto the subject 105 to be imaged. The array transducer 104 is attached to a motor 105, such a linear stepper motor, which moves the transducer 104 in predetermined increments along a desired path. A beamformer 106 is connected to elements of the active aperture of the array transducer 104, and is used to determine the aperture of the array transducer 104.

During transmission a laser from the fiber optical cable penetrates into the subject 105 and generates ultrasound signals from the tissues of the subject. The ultrasound signals are received by the elements of the active aperture of the array transducer 104 and converted into an analog electrical signal emanating from each element of the active aperture. The electrical signal is sampled to convert it from an analog to a digital signal in the beamformer 106. In some embodiments, the array transducer 104 also has a receive aperture that is determined by a beamformer control, which tells a receive beamformer which elements of the array to include in the active aperture and what delay profile to use. The receive beamformer can be implemented using at least one field programmable gate array (FPGA) device. The processing unit can also comprise a transmit beamformer, which may also be implemented using at least one FPGA device.

A central processing unit, e.g. a computer 101, has control software 109 that runs the components of the system, including the laser system 102 and transducer motor 105. The computer 101 also has software for processing received data, for example, using three-dimensional visualization software 108, to generate images based on the received ultrasound signals. The images are then displayed on a monitor 107 to be viewed by the user.

The components of the computer 101 can include, but are not limited to, one or more processors or processing units, a system memory, and a system bus that couples various system components including the beamformer 106 to the system memory. A variety of possible types of bus structures may be used, including a memory bus or memory controller, a peripheral bus, an accelerated graphics port, and a processor or local bus using any of a variety of bus architectures. By way of example, such architectures can include an Industry Standard Architecture (ISA) bus, a Micro Channel Architecture (MCA) bus, an Enhanced ISA (EISA) bus, a Video Electronics Standards Association (VESA) local bus, and a Peripheral Component Interconnects (PCI) bus also known as a Mezzanine bus. This bus, and all buses specified in this description can also be implemented over a wired or wireless network connection. This system can also be implemented over a wired or wireless network connection and each of the subsystems, including the processor, a mass storage device, an operating system, application software, data, a network adapter, system memory, an Input/Output Interface, a display adapter, a display device, and a human machine interface 102, can be contained within one or more remote computing devices at physically separate locations, connected through buses of this form, in effect implementing a fully distributed system.

The computer 101 typically includes a variety of computer readable media. Such media can be any available media that is accessible by the computer 101 and includes both volatile and non-volatile media, removable and non-removable media. The system memory includes computer readable media in the form of volatile memory, such as random access memory (RAM), and/or non-volatile memory, such as read only memory (ROM). The system memory typically contains data such as data and/or program modules such as operating system and application software that are immediately accessible to and/or are presently operated on by the processing unit.

The computer 101 may also include other removable/non-removable, volatile/non-volatile computer storage media. By way of example, a mass storage device which can provide non-volatile storage of computer code, computer readable instructions, data structures, program modules, and other data for the computer 101. For example, a mass storage device can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules can be stored on the mass storage device, including by way of example, an operating system and application software. Data including 3D images can also be stored on the mass storage device. Data can be stored in any of one or more databases known in the art. Examples of such databases include, DB2™, Microsoft™ Access, Microsoft™ SQL Server, Oracle™, mySQL, PostgreSQL, and the like. The databases can be centralized or distributed across multiple systems.

A user can enter commands and information into the computer 101 via an input device. Examples of such input devices include, but are not limited to, a keyboard, pointing device (e.g., a “mouse”), a microphone, a joystick, a serial port, a scanner, and the like. These and other input devices can be connected to the processing unit via a human machine interface that is coupled to the system bus, but may be connected by other interface and bus structures, such as a parallel port, game port, or a universal serial bus (USB). In an exemplary system of an embodiment according to the present invention, the user interface can be chosen from one or more of the input devices listed above. Optionally, the user interface can also include various control devices such as toggle switches, sliders, variable resistors and other user interface devices known in the art. The user interface can be connected to the processing unit. It can also be connected to other functional blocks of the exemplary system described herein in conjunction with or without connection with the processing unit connections described herein.

A display device or monitor 107 can also be connected to the system bus via an interface, such as a display adapter. For example, a display device can be a monitor or an LCD (Liquid Crystal Display). In addition to the display device 107, other output peripheral devices can include components such as speakers and a printer which can be connected to the computer 101 via Input/Output Interface.

The computer 101 can operate in a networked environment using logical connections to one or more remote computing devices. By way of example, a remote computing device can be a personal computer, portable computer, a server, a router, a network computer, a peer device or other common network node, and so on. Logical connections between the computer 101 and a remote computing device can be made via a local area network (LAN) and a general wide area network (WAN). Such network connections can be through a network adapter. A network adapter can be implemented in both wired and wireless environments. Such networking environments are commonplace in offices, enterprise-wide computer networks, intranets, and the Internet. The remote computer may be a server, a router, a peer device or other common network node, and typically includes all or many of the elements already described for the computer 101. In a networked environment, program modules and data may be stored on the remote computer. The logical connections include a LAN and a WAN. Other connection methods may be used, and networks may include such things as the “world wide web” or Internet.

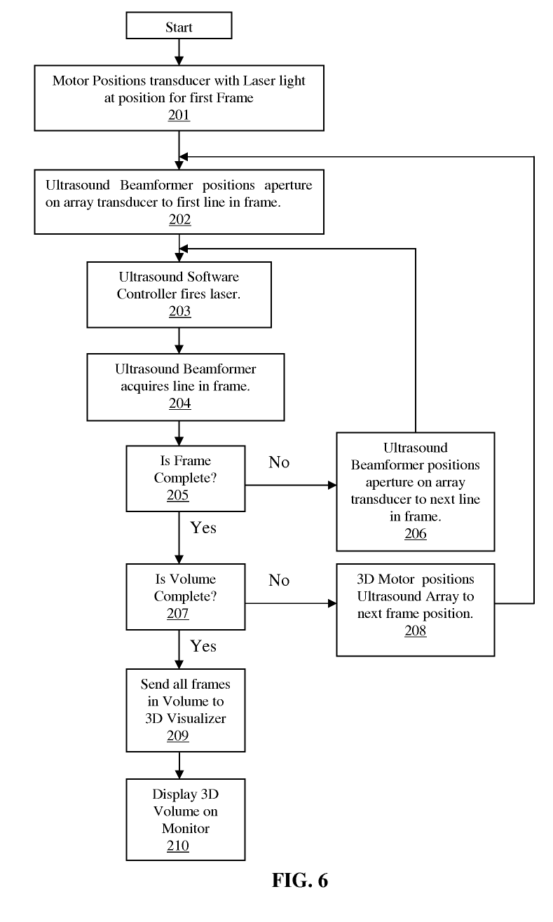

FIG. 6 is a block diagram showing a flow of operation for constructing a complete three-dimensional volume using a photoacoustic imaging system according to the present invention. In the first step (block 201), a motor moves an array transducer into position to obtain the first line of a frame. An ultrasound beamformer then positions the aperture on the array transducer for the first line in the frame (block 202). Ultrasound control software on a computer is used to fire the laser at the tissue of the subject to generate ultrasonic waves (block 203), and the ultrasound beamformer acquires the first line of the frame from the signals received by the array transducer (block 204).

Once the first line of the frame is acquired, the beamformer positions the aperture on the array transducer for the next line in the frame (block 206). The laser is fired again (block 203) and the ultrasound beamformer acquires the next line in the frame (block 204). This process continues until the frame is completed, i.e. the desired number of lines for the frame has been obtained (block 205).

The number of lines per frame can vary according the application, the parameters of the system, and/or requirements of the operator. Typically each frame has from about 10 to about 1024 lines, with 256 lines per frame or 512 lines per frame being suitable for many situations.

Once the first frame is completed, the motor moves the array transducer into position to obtain the second frame (block 208). The lines of the second frame are then acquired in the same fashion as for the first frame described above (blocks 202-206). Once the second frame is completed, the motor moves the array transducer into position to obtain another frame and so on until the desired number of frames has been acquired (block 207). All the frames are then processed by standard three-dimensional visualization software on the computer (block 209) to generate a three-dimensional image on a monitor (block 210). An example of three-dimensional volume image obtainable by this method is shown in FIG. 4.

Software on the computer allows the user to move and manipulate the image to provide various views, cross-sections, etc. of areas of interest. For example, the operator can rotate, and/or cut and slice into the cube to expose additional views of the imaged subject matter. Different rendering algorithms that are built into the software can be activated to help a user to visualize the anatomy of interest. 2D and volumetric measurement can then be performed on the volume.

The processing of the disclosed method can be performed by software components. The disclosed method may be described in the general context of computer-executable instructions, such as program modules, being executed by one or more computers or other devices. Generally, program modules include computer code, routines, programs, objects, components, data structures, etc. that perform particular tasks or implement particular abstract data types. The disclosed method may also be practiced in grid-based and distributed computing environments where tasks are performed by remote processing devices that are linked through a communications network. In a distributed computing environment, program modules may be located in both local and remote computer storage media including memory storage devices.

Aspects of the exemplary systems shown in the Figures and described herein can be implemented in various forms including hardware, software, and a combination thereof. The hardware implementation can include any or a combination of the following technologies, which are all well known in the art: discrete electronic components, a discrete logic circuit(s) having logic gates for implementing logic functions upon data signals, an application specific integrated circuit having appropriate logic gates, a programmable gate array(s) (PGA), field programmable gate array(s) (FPGA), etc. The software comprises an ordered listing of executable instructions for implementing logical functions, and can be embodied in any computer-readable medium for use by or in connection with an instruction execution system, apparatus, or device, such as a computer-based system, processor-containing system, or other system that can fetch the instructions from the instruction execution system, apparatus, or device and execute the instructions.

The photoacoustic imaging systems and methods of the invention can be used in a wide variety of clinical and research applications to image various tissues, organs, (e.g., heart, kidney, brain, liver, blood, etc.) and/or disease conditions of a subject. For example, the described embodiments enable in vivo visualization, assessment, and measurement of anatomical structures and hemodynamic function in longitudinal imaging studies of small animals. The systems can provide images having very high resolution, image uniformity, depth of field, adjustable transmit focal depths, multiple transmit focal zones for multiple uses. For example, the photoacoustic image can be of a subject or an anatomical portion thereof, such as a heart or a heart valve. The image can also be of blood and can be used for applications including evaluation of the vascularization of tumors. The systems can be used to guide needle injections.

For imaging of small animals, it may be desirable for the transducer to be attached to a fixture during imaging. This allows the operator to acquire images free of the vibrations and shaking that usually result from “free hand” imaging. The fixture can have various features, such as freedom of motion in three dimensions, rotational freedom, a quick release mechanism, etc. The fixture can be part of a “rail system” apparatus, and can integrate with the heated mouse platform. A small animal subject may also be positioned on a heated platform with access to anesthetic equipment, and a means to position the transducer relative to the subject in a-flexible manner.

The systems can be used with platforms and apparatus used in imaging small animals including “rail guide” type platforms with maneuverable probe holder apparatuses. For example, the described systems can be used with multi-rail imaging systems, and with small animal mount assemblies as described in U.S. patent application Ser. No. 10/683,168, entitled “Integrated Multi-Rail Imaging System,” U.S. patent application Ser. No. 10/053,748, entitled “Integrated Multi-Rail Imaging System,” U.S. patent application Ser. No. 10/683,870, now U.S. Pat. No. 6,851,392, issued Feb. 8, 2005, entitled “Small Animal Mount Assembly,” and U.S. patent application Ser. No. 11/053,653, entitled “Small Animal Mount Assembly,” each of which is fully incorporated herein by reference.

Small animals can be anesthetized during imaging and vital physiological parameters such as heart rate and temperature can be monitored. Thus, an embodiment of the system may include means for acquiring ECG and temperature signals for processing and display. An embodiment of the system may also display physiological waveforms such as an ECG, respiration or blood pressure waveform

The described embodiments can also be used for human clinical, medical, manufacturing (e.g., ultrasonic inspections, etc.) or other applications where producing a three-dimensional photoacoustic image is desired.

As used in this description and in the following claims, “a” or “an” means “at least one” or “one or more” unless otherwise indicated. In addition, the singular forms “a”, “an”, and “the” include plural referents unless the content clearly dictates otherwise. Thus, for example, reference to a composition containing “a compound” includes a mixture of two or more compounds.

As used in this specification and the appended claims, the term “or” is generally employed in its sense including “and/or” unless the content clearly dictates otherwise.

The recitation herein of numerical ranges by endpoints includes all numbers subsumed within that range (e.g. 1 to 5 includes 1, 1.5, 2, 2.75, 3, 3.80, 4, and 5).

Unless otherwise indicated, all numbers expressing quantities of ingredients, measurement of properties and so forth used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings of the present invention. At the very least, and not as an attempt to limit the scope of the claims, each numerical parameter should at least be construed in light of the number of reported significant digits and by applying ordinary rounding techniques. Any numerical value, however, inherently contains certain errors necessarily resulting from the standard deviations found in their respective testing measurements.

Various modifications and alterations to the invention will become apparent to those skilled in the art without departing from the scope and spirit of this invention. It should be understood that the invention is not intended to be unduly limited by the specific embodiments and examples set forth herein, and that such embodiments and examples are presented merely to illustrate the invention, with the scope of the invention intended to be limited only by the claims attached hereto.

The complete disclosures of the patents, patent documents, and publications cited herein are hereby incorporated by reference in their entirety as if each were individually incorporated.

What is claimed is:

1. A photoacoustic imaging system for obtaining two-dimensional (2D) or three-dimensional (3D) images of a target, said system comprising:
(a) an ultrasound transducer for receiving ultrasound waves,
(b) a laser system for generating pulses of non-ionizing laser light, and
(c) a fiber optic cable comprising a plurality of optical fibers for directing the laser light to the target, wherein the plurality of optical fibers are attached to the transducer.

2. The system of claim 1, wherein the ultrasound transducer is an arrayed transducer comprising a plurality of transducer elements for generating and receiving ultrasound waves and the plurality of optical fibers are attached to the plurality of transducer elements.

3. The system of claim 2, wherein the arrayed transducer is selected from the group consisting of a linear array transducer, a phased array transducer, a two-dimensional array transducer, and a curved array transducer.

4. The system of claim 3, wherein the arrayed transducer is a linear array transducer.

5. The system of claim 1, further comprising a motor for moving the ultrasound transducer.

6. The system of claim 5, wherein the motor is a linear stepper motor for moving the transducer along a linear path to collect a series of frames separated by a predetermined step size.

7. The system of claim 6, wherein the predetermined step size may be adjusted by a user.

8. The system of claim 7, wherein the predetermined step size is at least 10 μm.

9. The system of claim 1, further comprising a beamformer for receiving ultrasound signals from the transducer and focusing them along an ultrasound line.

10. The system of claim 9, wherein the optical fibers are positioned on the transducer so that the laser light delivered to a subject is aligned with the ultrasound line.

11. The system of claim 1, wherein the laser light is capable of generating ultrasound signals within the tissue of a subject, and the optical fibers are arranged on the transducer so that each ultrasound line within a scan plane receives about the same level of laser light intensity.

12. The system of claim 1, further comprising a computer for controlling system components and processing received ultrasound data into an image, and a monitor for displaying the image.

13. The system of claim 12, wherein the image comprises three-dimensional (3D) volume data.

14. The system of claim 12, wherein the computer system has software for visualizing received ultrasound data.

15. A method of generating a three-dimensional (3D) photoacoustic image of a subject, said method comprising the steps of:
(a) delivering laser radiation to a region of tissue within the subject to generate ultrasound signals for a frame;
(b) detecting the ultrasound signals for the frame;
(c) delivering laser radiation to an adjacent region of tissue to generate ultrasound signals for a next frame;
(d) detecting the ultrasound signals for the next frame;
(e) repeating steps (c) and (d) to generate a series of consecutive frames;
(f) stacking the series of consecutive frames to generate a three-dimensional volume of data; and
(g) displaying a three-dimensional image generated from the volume of data on a monitor.

16. The method of claim 15, wherein the ultrasound signals are detected using an ultrasound transducer and the laser radiation is delivered via at least one optical fiber attached to the transducer.

17. The method of claim 16, wherein the ultrasound transducer is a linear array transducer.

18. The method of claim 17, wherein the ultrasound signals for the frame are generated by a method comprising the steps of:
(i) positioning an aperture on the array transducer to a first line in the frame;
(ii) delivering laser radiation to the subject for the first line in the frame;
(iii) acquiring ultrasound signals for the first line in the frame;
(iv) positioning the aperture on the array transducer to a next line in the frame;
(v) delivering laser radiation to the subject for the next line in the frame;
(vi) acquiring ultrasound signals for the next line in the frame; and
(vii) repeating steps (iv) through (vi) for each subsequent line in the frame until a desired number of lines for the frame have been acquired.

19. The method of claim 18, wherein a beamformer is used to position the aperture on the array transducer.

20. The method of claim 19, wherein the number of lines for the frame is from about 10 to about 1024.

21. The method of claim 20, wherein the number of lines for the frame is 256.

22. The method of claim 17, wherein the linear array transducer is attached to a motor for controlled movement of the transducer along a desired path.

23. The method of claim 22, wherein the motor moves the transducer from a first position to acquire data for the frame to a second position to acquire data for the adjacent frame.

24. The method of claim 15, wherein the subject is a small animal.

25. The method of claim 24, wherein the subject is a rat.

26. The method of claim 25, wherein the subject is a mouse.

27. The method of claim 26, further comprising imaging an organ of the subject.

28. The method of claim 27, wherein the organ is selected from a heart, kidney, brain, liver, and blood.

29. The method of claim 15, further comprising imaging a neo-plastic condition of the subject.

30. A photoacoustic imaging system for obtaining two-dimensional (2D) or three-dimensional (3D) images of a target, said system comprising:
(a) a scan head having a moving support arm,
(b) an ultrasound transducer for receiving ultrasound waves, wherein the transducer is located at an end of the support arm which moves the transducer along a scan plane;
(c) a laser system for generating pulses of non-ionizing laser light, and
(d) at least one optical fiber for directing the laser light to a target, wherein the optical fiber is attached to the transducer.

31. The system of claim 30, comprising a plurality of optical fibers attached to the transducer.

32. The system of claim 30, wherein the ultrasound transducer is further capable of generating ultrasound at a frequency of at least 20 MHz.

33. The system of claim 30, further comprising a motor for moving the transducer in a plane perpendicular to the scan plane.

34. The system of claim 30, further comprising a computer for controlling system components and processing received ultrasound data into an image, and a monitor for displaying the image.

35. The system of claim 34, wherein the image comprises three-dimensional (3D) volume data.

36. The system of claim 34, wherein the computer system has software for visualizing received ultrasound data.
